(19) United States
(12) Patent Application Publication
(10) Pub. No.: US 2007/0123538 A1
(43) Pub. Date: May 31, 2007

(54) Compositions comprising a combination of CCR5 and CXCR4 antagonists

(75) Inventors: Lisa Dunkle, Jersey City, NJ (US); Julie M. Strizki, Yardley, PA (US); Bahige M. Baroudy, Westfield, NJ (US); Jayaram R. Tagat, Westfield, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/605,124

(22) Filed: Nov. 28, 2006

Related U.S. Application Data
(60) Provisional application No. 60/740,861, filed on Nov. 30, 2005.

(51) Int. Cl. A61K 31/506

(52) U.S. Cl. 514/252.18; 514/253.13

(57) ABSTRACT

A composition including a CXCR4 antagonist and a CCR5 antagonist represented by formula I or II: 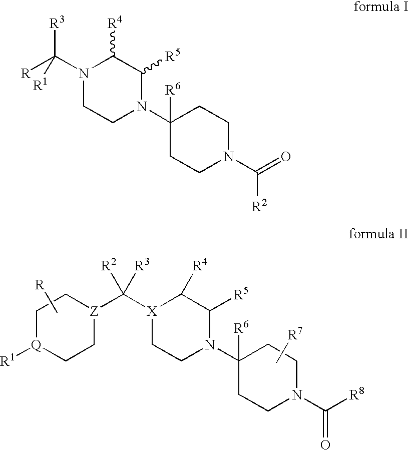 or an acceptable salt, solvate or ester thereof. The CXCR4 antagonist includes at least one of AMD-070, CS-3955, KRH-1120, KRH-2731, and KRH-1636.

This Application claims the benefit of U.S. Provisional Application Ser. No. 60/740,861 filed Nov. 30, 2005, which is incorporated herein by reference in its entirety.

### DESCRIPTION OF THE DISCLOSURE

1. Field of the Disclosure

The present invention relates to a combination comprising a CCR5 antagonist, such as a compound of formula I or II, and a CXCR4 antagonist, such as AMD-070, CS-3955, KRH-1120, KRH-2731, KRH-1636. Also, disclosed is a pharmaceutical composition comprising a CCR5 antagonist and a CXCR4 antagonist. Further, there are disclosed methods of treatment comprising administering the disclosed pharmaceutical composition, and a kit.

2. Background of the Disclosure

The global health crisis caused by HIV, the causative agent of Acquired Immunodeficiency Syndrome (AIDS), is unquestioned, and while recent advances in drug therapies have been successful in slowing the progression of AIDS, there is still a need to find a safer, more and to control the vir

It has been reported that the CCR5 gene plays a role in resistance to HIV infection. HIV infection begins by attachment of the virus to a target cell membrane through interaction with the cellular receptor CD4 and a secondary chemokine co-receptor molecule, and proceeds by replication and dissemination of infected cells through the blood and other tissues. Among the molecules in the chemokine receptor family, the CCR5 and CXCR4 receptors are known to act as coreceptors for HIV infenction in vivo. Clinically studies have recently demonstrated that small molecule agents that bind to the viral co-receptors CCR5 and CXCR4 and HIV can interfere with HIV infection and reduce HIV RNA titers in infected patients. These agents may prove useful as therapeutics for HIV treatment.

The present invention relates to small molecules which are CCR5 antagonists and CXCR4 antagonists.

Related piperazine derivatives which are muscarinic antagonists useful in the treatment of cognitive disorders such as Alzheimer's disease are disclosed in U.S. Pat. Nos. 5,883,096; 6,037,352; 5,889,006.

A-M. Vandamme et al., Antiviral Chemistry & Chemotherapy, 9:187-203 (1998) disclose current clinical treatments of HIV-1 infections in man including at least triple drug combinations or so-called Highly Active Antiretroviral Therapy (“HAART”); HAART involves various combinations of nucleoside reverse transcriptase inhibitors (“NRTI”), non-nucleoside reverse transcriptase inhibitors (“NNRTI”) and HIV protease inhibitors (“PI”). In adherent drug-naive patients, HAART is effective in reducing mortality and progression of HIV-1 to AIDS. However, these multidrug therapies do not eliminate HIV-1 and long-term treatment usually results in multidrug resistance. Development of new drug therapies to provide better HIV-1 treatment remains a priority.

John Moore et al (see Journal of Virology, Vol. 74, No. 5, 6893-6910 (2000), and Vol. 73, No. 4, 3443-3448 (1999), have used coreceptor-targeted inhibitors to investigate which coreceptors are used by human immunodeficiency virus type 1 (HIV-1), simian immunodeficiency viruses (SIV), and human inmmunodeficiency virus type 2 (HIV-2) to enter peripheral blood mononuclear cells (PBMC). The inhibitors used were TAK-779, which is specific for CCR5 and CCR2, aminooxypentane-RANTES, which blocks entry via CCR5 and CCR3 and AMD 3100, which targets CXCR4. It was found that for all of the HIV-1 isolates and all but one of the HIV-2 isolates tested, the only relevant coreceptors were CCR5 and CXCR4.

U.S. Patent Application Publication US 2005/0165063 AA1 refers to low-molecular weight drugs which have CXCR4 antagonism.

### SUMMARY OF THE DISCLOSURE

In accordance with the disclosure, there is disclosed a composition comprising at least one CCR5 antagonist and at least one CXCR4 antagonist. In one embodiment, the CXR4 antagonist compound is at least one of AMD-0700, CS-3955, KRH-1120, KRH-2731, and KRH-1636.

In one embodiment, the CCR5 antagonist compound is a compound of formula I 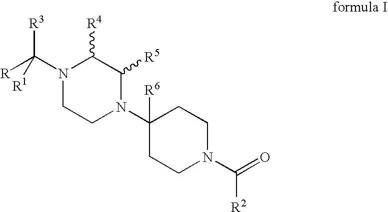
or a pharmaceutically acceptable salt or solvate thereof,

wherein R is optionally substituted phenyl, pyridyl, thiophenyl or naphthyl;

R¹ is hydrogen or alkyl;

R² is substituted phenyl, substituted heteroaryl, naphthyl, fluorenyl, diphenylmethyl or optionally substituted phenyl- or heteroaryl-alkyl;

R³ is hydrogen, alkyl, alkoxyalkyl, cycloalkyl, cycloalkylalkyl, or optionally substituted phenyl, phenylalkyl, naphthyl, naphthylalkyl, heteroaryl or heteroarylalkyl;

R⁴, R⁵ and R⁷ are hydrogen or alkyl; and

R⁶ is hydrogen, alkyl or alkenyl.

In another embodiment, the CCR5 antagonist is a compound of formula II 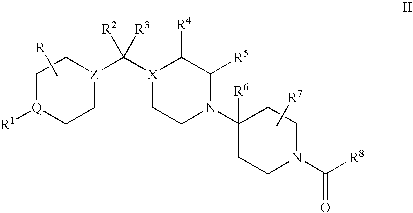
or a pharmaceutically acceptable salt, solvate, or ester thereof, wherein:

Q, X and Z are independently selected from the group consisting of CH and N, provided that one or both of Q and Z is N;

R, R⁴, R⁵, R⁶ and R⁷ are independently selected from the group consisting of H and (C₁-C₆)alkyl;

R¹ is H, (C₁-C₆)alkyl, fluoro-(C₁-C₆)alkyl-, R⁹-aryl(C₁-C₆)alkyl-, R⁹-heteroaryl-(C₁-C₆)alkyl-, (C₁-C₆)alkyl-SO₂—, (C₃-C₆)cycloalkyl-SO₂—, fluoro-(C₁-C₆)alkyl-SO₂—, R⁹-aryl-SO₂—, R⁹-heteroaryl-SO₂—, N(R²²)(R²³)-SO₂—, (C₁-C₆)alkyl-C(O)—, (C₃-C₆)cyclo-alkyl-C(O)—, fluoro-(C₁-C₆)alkyl-C(O)—, R⁹-aryl-C(O)—, NH—(C₁-C₆)alkyl-C(O)— or R⁹-aryl-NH—C(O)—;

R² is H or (C₁-C₆)alkyl, and R³ is H, (C₁-C₆)alkyl, (C₁-C₆)alkoxy(C₁-C₆)alkyl-, (C₃-C₁₀)-cycloalkyl-, (C₃-C₁₀)cycloalkyl(C₁-C₆)alkyl-, R⁹-aryl, R⁹-aryl(C ₁-C₆)-alkyl-, R⁹-heteroaryl, or R⁹-heteroaryl(C₁-C₆)alkyl-, provided that both X and Z are not each N;

or R² and R³ together are ═O, ═NOR¹⁰, ═N—NR¹¹R¹² or ═CH(C₁-C₆)alkyl, provided that when one or both of X and Z is N, R² and R³ together are not ═CH(C₁-C₆)alkyl;

and when X and Z are each CH, R³ can also be (C₁-C₆)alkoxy, R⁹-aryloxy, R⁹-heteroaryloxy, (C₁-C₆)alkyl-C(O)O—, (C₁-C₆)alkyl-NH—C(O)O—, N((C₁-C₆)alkyl)₂—C(O)O—, (C₁-C₆)alkyl-C(O)—NR¹³—, (C₁-C₆)alkyl-O—C(O)—NR¹³—, (C₁-C₆)alkyl-NH—C(O)—NR¹³— or N((C₁-C₆)alkyl)₂—C(O)— NR¹³—;

R⁸ is (R¹⁴,R¹⁵,R¹⁶)-substituted phenyl, (R¹⁴,R¹⁵,R¹⁶)-substituted 6-membered heteroaryl, (R¹⁴, R¹⁵,R¹⁶)-substituted 6-membered heteroaryl N-oxide, (R¹⁷, R¹⁸)-substituted 5-membered heteroaryl, naphthyl, fluorenyl,
diphenylmethyl,

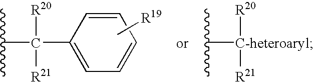

R⁹ is 1, 2 or 3 substituents independently selected from the group consisting of H, halogen, (C₁-C₆)alkyl, (C₁-C₆)alkoxy, —CF₃, —OCF₃, CH₃C(O)—, —CN, CH₃SO₂—, CF₃SO₂— and —N(R²²)(R²³);

R¹⁰ is H, (C₁-C₆)alkyl, fluoro(C₁-C₆)alkyl-, (C₃-C₁₀)cycloalkyl(C₁-C₆)alkyl-, hydroxy(C₂-C₆)alkyl-, (C₁-C₆)alkyl-O—(C₂-C₆)alkyl-, (C₁-C₆)alkyl-O—C(O)—(C₁-C₆)alkyl- or N(R²²)(R²³)—C(O)—(C₁-C₆)alkyl-;

R¹¹ and R¹² are independently selected from the group consisting of H, (C₁-C₆)alkyl and (C₃-C¹⁰)cycloalkyl, or R¹¹ and R¹² together are C₂-C₆alkylene and form a ring with the nitrogen to which they are attached;

R¹⁴ and R¹⁵ are independently selected from the group consisting of (C₁-C₆)alkyl, halogen, —NR²²R²³, —OH, —CF₃, —OCH₃, —O-acyl and —OCF₃;

R¹⁶ is R¹⁴, hydrogen, phenyl, —NO₂, —CN, —CH₂F, —CHF₂, —CHO, —CH═NOR²⁴, pyridyl, pyridyl N-oxide, pyrimidinyl, pyrazinyl, —N(R²⁴)CONR²⁵R²⁶, —NHCONH(chloro-(C₁-C₆)alkyl), —NHCONH((C₃-C₁₀)cycloalkyl(C₁-C₆)alkyl), —NHCO(C₁-C₆)alkyl, —NHCOCF₃, —NHSO₂N(R²²)(R²³), —NHSO₂(C₁-C₆)alkyl, —N(SO₂CF₃)₂, —NHCO₂—(C₁-C₆)alkyl, C₃-C₁₀ cycloalkyl, —SR²⁷, —SOR²⁷, —SO₂R²⁷, —SO₂NH(R²²), —OSO₂(C₁-C₆)alkyl, —OSO₂CF₃, hydroxy(C₁-C₆)alkyl-, —CON R²⁴R²⁵, —CON(CH₂CH₂OCH₃)₂, —OCONH(C₁-C₆)alkyl, —CO₂R²⁴, —Si(CH₃)₃ or —B(OC(CH₃)₂)₂;

R¹⁷ is (C₁-C₆)alkyl, —N(R²²)(R²³) or R¹⁹-phenyl;

R¹³, R¹⁸, R²², R²³, R²⁴, R²⁵ and R²⁶ are independently selected from the group consisting of H and (C₁-C₆)alkyl;

R¹⁹ is 1, 2 or 3 substituents independently selected from the group consisting of H, (C₁-C₆)alkyl, —CF₃, —CO₂R²⁵, —CN, (C₁-C₆)alkoxy and halogen;

R²⁰ and R²¹ are independently selected from the group consisting of H and

(C₁-C₆)alkyl, or R²⁰ and R²¹ together with the carbon to which they are attached form a spiro ring of 3 to 6 carbon atoms; and

R²⁷ is (C₁-C₆)alkyl or phenyl.

In another embodiment the compound of formula I is a compound of formula III 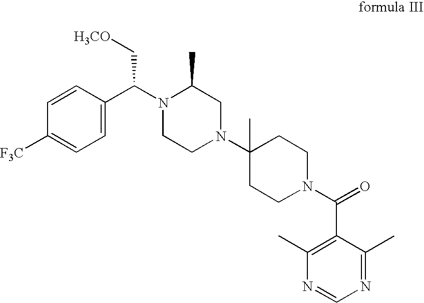
or a pharmaceutically acceptable salt or solvate thereof.

In another embodiment, the compound of formula II is a compound of formula IV: 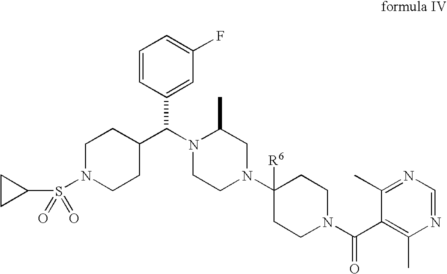
or a pharmaceutically acceptable salt or solvate thereof.

In another embodiment, the compound of formula II is a compound of formula V: 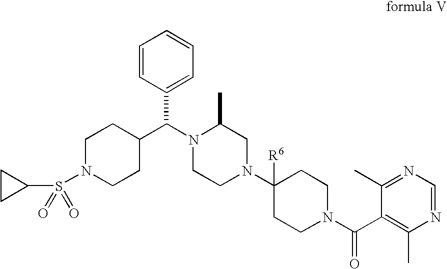
or a pharmaceutically acceptable salt of solvate thereof.

Another aspect of the invention is a pharmaceutical composition comprising an effective amount of at least one CCR5 antagonist of formula I-V and an effective amount of at least one CXCR4 antagonist compound in combination with a pharmaceutically acceptable carrier. Another aspect of the invention is a pharmaceutical composition for treatment of solid organ transplant rejection, graft v. host disease, arthritis, rheumatoid arthritis, inflammatory bowel disease, atopic dermatitis, psoriasis, asthma, allergies or multiple sclerosis comprising an effective amount of at least one CCR5 antagonist compound of formula I-V and at least one CXCR4 antagonist compound in combination with a pharmaceutically acceptable carrier.

Yet another aspect of this invention is a method of treatment of HIV comprising administering to a human in need of such treatment an effective amount of at least one CCR5 antagonist compound of formula I-V and an effective amount of at least one CXCR4 antagonist compound. Another aspect of the invention is a method of treatment of solid organ transplant rejection, graft v. host disease, arthritis, rheumatoid arthritis, inflammatory bowel disease, atopic dermatitis, psoriasis, asthma, allergies or multiple sclerosis comprising administering to a human in need of such treatment an effective amount of at least one CCR5 antagonist compound of formula I-V and at least one CXCR4 antagonist compound.

Still another aspect of this invention is the use of at least one CCR5 antagonist of formula I-V and at least one CXCR4 antagonist compound in combination with one or more antiviral or other agents useful in the treatment of HIV infection. Still another aspect of this invention is the use of at least one CCR5 antagonist of formula I-V and at least one CXCR4 antagonist compound in combination with one or more other agents useful in the treatment of solid organ transplant rejection, graft v. host disease, inflammatory bowel disease, rheumatoid arthritis or multiple sclerosis. The CCR5 and CXCR4 antagonist compounds and antiviral or other agents can be administered in a single dosage form or they can be administered separately; a kit comprising separate dosage forms of the actives is also contemplated.

It is to be understood that both the foregoing general description and the following detailed description are exemplary and explanatory only and are not restrictive of the disclosure, as claimed.

### DESCRIPTION OF THE EMBODIMENTS

As used herein, the following terms are used as defined below unless otherwise indicated.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbomyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkyiheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —C(═N—CN)—NH₂, —C(═NH)—NH₂, —C(═NH)—NH(alkyl), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

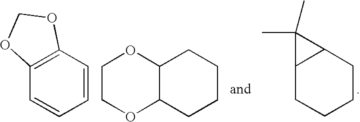

Optionally substituted ring system such as “optionally substituted phenyl”, “optionally substituted heteroaryl” etc., refers to a ring system which are optionally substituted with one or more “ring system substitutent” as set forth above. Similarly, “substituted phenyl”, and “substituted heteroaryl” refer to a phenyl and a heteroaryl group respectively that is substituted with one or more “ring system substitutent” as set forth above.

The term “viral infection” is used to describe a diseased state, which can be latent, where a virus invades a cell, uses the cell's reproductive machinery to multiply or replicate, and ultimately releases progeny virus particles followed by further infection of other cells by the progeny.

The terms “treating” or “preventing” used in relation to a viral infection means to inhibit viral activity, expression, replication or transmission of a virus, or to prevent the virus from establishing itself in a host cell, and which results in an amelioration or alleviation of the symptoms of the disease caused by the viral infection. Such prevention includes the prevention of an infection after exposure (i.e., prophylaxis). A treatment or therapy is considered therapeutic if there is a reduction in viral load or decrease in mortality or morbidity.

A “therapeutically effective amount” of a CXCR4 antagonist compound or a CCR5 antagonist compound, or their derivatives, is an amount sufficient to treat or prevent a viral infection and according to a suitable administration schedule, i.e., the amount and dosaging schedule exhibits antiviral activity, thereby lowering HIV RNA plasma levels in the serum of an infected individual to less than 500 copies per ml of serum, preferably to less than 200 copies per ml of serum, more preferably to less than 50 copies per ml of serum, and most preferably the number of copies is undetectable, as measured by quantitative, multi-cycle reverse transcriptase PCR methodology. HIV RNA is preferably measured using the methodology of Amplicor-1 Monitor 1.5 (available from Roche Diagnostics) or of Nuclisens HIV-1 QT-1.

The term “combination therapy” refers to a therapy for treating viral infections, preferably HIV, which includes administration of an effective amount of a CCR5 antagonist and a CXCR4 antagonist compound. A combination therapy of this invention may include one or more antiviral agents, e.g., HAART. In addition, a combination therapy of this invention can be used as a prophylactic measure in previously uninfected individuals after a possible acute exposure to an HIV virus. Examples of such prophylactic use of the compounds may include, but are not limited to, prevention of virus transmission from mother to infant and other settings where the likelihood of HIV transmission exists, such as, for example, accidents in health care settings wherein workers are exposed to HIV-containing blood products. Moreover, a combination therapy of this invention can be used as a prophylactic measure in previously uninfected individuals, but those at a high risk of exposure as either a systemic therapy or as topical microbicide in high risk individuals.

The term “synergistic” refers to a combination which is more effective than the additive effects of any two or more single agents. A “synergistic effect” refers to the ability to use lower amounts or dosages of antiviral agents in a single therapy to treat or prevent viral infection. The lower doses typically result in a decreased toxicity without reduced efficacy. In addition, a synergistic effect can improve efficacy, e.g., improved antiviral activity, or avoid or reduce the extent of any viral resistance against an antiviral agent. A synergistic effect between a CXCR4 antagonist compound, or a pharmaceutically acceptable derivative thereof, and a CCR5 antagonist compound, or a pharmaceutically acceptable salt thereof, can be determined from conventional antiviral assays, e.g., as described infra. The results of an assay can be analyzed using Chou and Talalay's combination method to obtain a Combination Index (Chou and Talalay, 1984, Adv. Enzyme Regul. 22:27-55) and ‘Dose Effect Analysis with Microcomputers’ software (Chou and Chou, 1987, Software and Manual. p19-64. Elsevier Biosoft, Cambridge, UK). A Combination Index value of less than 1 indicates synergy, greater than 1 indicates antagonism and equal to 1 indicates an additive effect. The results of these assays can also be analyzed using the method of Pritchard and Shipman (Pritchard and Shipman, 1990, Antiviral Research 14:181-206).

The term “pharmaceutically acceptable carrier” refers to a carrier medium that does not interfere with the effectiveness of the biological activity of the active ingredient, is chemically inert and is generally not toxic to the recipient.

The term “pharmaceutically acceptable derivative” refers to a truncation, analog or other modification of a polypeptide, which exhibits antiviral activity and is generally non-toxic.

The term “antiviral activity” refers to an inhibition of HIV transmission to uninfected CD4⁺cells, inhibition of the replication of HIV, prevention of HIV from establishing itself in a host, or ameliorating or alleviating the symptoms of the disease caused by HIV infection. These effects can be evidenced by a reduction in viral load or decrease in mortality and/or morbidity, which assays are described infra. An antiviral agent, or anti-HIV-1 drug, has antiviral activity and is useful for treating HIV-1 infections alone, or as part of a multi-drug combination therapy, e.g., the HMRT triple and quadruple combination therapies.

A “therapeutic agent” is any molecule, compound or therapy that improves the treatment of a viral infection or the diseases caused thereby. Preferably, the therapeutic agent has antiviral activity.

The terms “CCR5 antagonist compound” and “CCR5 antagonists” as used herein mean any compound that interferes with the interaction between the viral receptor CCR5 and HIV-1 to block entry of HIV-1 into the cell. Assays, e.g., the CCR5 Membrane Binding Assay, the HIV-1 Entry and the HIV-1 Entry Replication Assays, are presented herein to identify a compound as a CCR5 antagonist and to determine its CCR5 antagonist activity.

CCR5 Membrane Binding Assay

A high throughput screen utilizing a CCR5 membrane binding assay identifies inhibitors of RANTES binding. This assay utilizes membranes prepared from NIH 3T3 cells expressing the human CCR5 chemokine receptor which have the ability to bind to RANTES, a natural ligand for the receptor. Using a 96-well plate format, membrane preparations are incubated with ¹²⁵I-RANTES in the presence or absence of compound for one hour. Compounds are serially diluted over a wide range of 0.001 ug/ml to 1 ug/ml and tested in triplicates. Reaction cocktails are harvested through glass fiber filters, and washed thoroughly. Total counts for replicates are averaged and data reported as the concentration required to inhibit 50 percent of total ¹²⁵I-RANTES binding. Compounds with potent activity in the membrane binding assay are further characterized in secondary cell-based HIV-1 entry and replication assays.

HIV-1 Entry Assay

Replication defective HIV-1 reporter virions are generated by cotransfection of a plasmid encoding the NL4-3 strain of HIV-1 (which has been modified by mutation of the envelope gene and introduction of a luciferase reporter plasmid) along with a plasmid encoding one of several HIV-1 envelope genes as described by Connor et al, Virology, 206 (1995), p. 935-944. Following transfection of the two plasmids by calcium phosphate precipitation, the viral supernatants are harvested on day 3 and a functional viral titer determined. These stocks are then used to infect U87 cells stably expressing CD4 and the chemokine receptors CCR5 or CXCR4 which have been preincubated with or without test compound. Infections are carried out for 2 hours at 37° C., the cells washed and media replaced with fresh media containing compound. The cells are incubated for 3 days, lysed and luciferase activity determined. Results are reported as the concentration of compound required to inhibit 50% of the luciferase activity in the control cultures.

HIV-1 Replication Assay

This assay uses primary peripheral blood mononuclear cells or the stable U87-CCR5 or U87-CXCR4 cell lines to determine the effect of compounds to block infection of primary HIV-1 strains. The primary lymphocytes are purified from normal healthy donors and stimulated in vitro with PHA and IL-2 three days prior to infection. Using a 96-well plate format, cells are pretreated with drug for 1 hour at 37° C. and subsequently infected with an CCR5 or CXCR4-tropic HIV-1 isolates. Following infection, the cells are washed to remove residual inoculum and cultured in the presence of compound for 4 days. Culture supernatants are harvested and viral replication measured by determination of viral p24 antigen concentration.

The terms “CXCR4 antagonist compound” and “CXCR4 antagonists” as used herein mean any compound that interferes with the interaction between the viral receptor CXCR4 and HIV-1 to block entry into the cell. Non-limiting examples of assays, including the HIV-1 Entry Assay and HIV-1 Replication Assay, are presented herein to identify a compound as a CXCR4 antagonist and to determine its CXCR4 antagonist activity.

A Calcium Flux Assay

Cells expressing the CXCR4 receptor can be loaded with calcium sensitive dyes prior to addition of the compound or the natural CXCR4 ligand. Compounds with agonist properties can induce a calcium flux signal in the cell, while CXCR4 antagonist are identified as compounds which do not induce signaling by themselves but are capable of blocking signaling by the natural ligand. See D. Schols, et al., “Inhibition of T-tropic HIV Strains by Selective Antagonization of the Chemokine Receptor CXCR4,” J. Exp. Med., 186(8):1383-1388 (1997).

Inhibition of Antibody Binding Assay

A CXC-chemokine can be added to SUP-T1 cells at certain concentrations for 15 mins. on ice or at room temperature. A 12G5 mAb can be added for 30 min. at room temperature. The cells can be washed, incubated with fluorescein isothiocyanate-conjugated goat-anti-mouse antibody, washed again, and analyzed by flow cytometry. The CXC-chemokine can be shown to inhibit the binding of the mAb to the CXCR4 receptor on the SUPT1 cells. See D. Schols, et al., “Bicyclams, a Class of Potent Anti-HIV agents, are Targeted at the HIV Coreceptor Fusin/CXCR4, ” Antiviral Research, 35:147-156 (1997).

The term “patients having HIV-1 infections” as used herein means any patient-including a pediatric patient-having HIV-1 infection and includes treatment-naive patients and treatment-experienced patients having the HIV-1 infection as well as treatment-naive patients and treatment-experienced patients co-infected with the HIV-1 and hepatitis C virus (“HCV”).

The term “pediatric patient” as used herein means a patient below the age of 17, and normally includes those from birth to 16 years of age.

The term “treatment-naive patients” as used herein means patients having HIV-1 or co-infected with the HIV-1 and HCV who have never been treated with any CCR5 antagonist compound or any CXCR4 antagonist compound.

The term “treatment-experienced” patients as used herein means those patients having HIV-1 or co-infected with the HIV-1 and HCV who have initiated some form of anti HIV therapy including, but not limited to HAART or some form of anti-HCV therapy, including but not limited to any CCR5 antagonist compound or any CXCR4 antagonist compound.

The term “patients having hepatitis C infections” as used herein means any patient-including a pediatric patient-having hepatitis C and includes treatment-naive patients having hepatitis C infections and treatment-experienced patients having hepatitis C infections as well as those pediatric, treatment-naive and treatment-experienced patients having chronic hepatitis C infections.

These patients having hepatitis C include those who are infected with multiple HCV genotypes including type 1 as well as those infected with, e.g., HCV genotypes 2, 3, 4, 5 and/or 6 and other possible HCV genotypes.

The term “treatment-naive patients having hepatitis C infections” as used herein means patients with hepatitis C who have never been treated with any CCR5 antagonist compound or any CXCR4 antagonist compound.

The term “treatment-experienced patients having hepatitis C infections” as used herein means patients with hepatitis C who have been treated with any CCR5 antagonist compound or any CXCR4 antagonist compound, including relapsers and non-responder.

The term “relapsers” as used herein means treatment-experienced patients with hepatitis C who have relapsed after initial response to previous treatment with any CCR5 antagonist compound or any CXCR4 antagonist compound.

The term “non-responders” as used herein means treatment-experienced patients with hepatitis C who have not responded to prior treatment with any CCR5 antagonist compound or any CXCR4 antagonist compound.

The term “nucleoside and nucleotide reverse transcriptase inhibitors” (“NRTI”s) as used herein means nucleosides and nucleotides and analogues thereof that inhibit the activity of HIV-1 reverse transcriptase, the enzyme which catalyzes the conversion of viral genomic HIV-1 RNA into proviral HIV-1 DNA.

The term “non-nucleoside reverse transcriptase inhibitors” (“NNRTI”s) as used herein means non-nucleosides that inhibit the activity of HIV-1 reverse transcriptase.

The term “protease inhibitor” (“PI”) as used herein means inhibitors of the HIV-1 protease, an enzyme required for the proteolytic cleavage of viral polyprotein precursors (e.g., viral GAG and GAG Pol polyproteins), into the individual functional proteins found in infectious HIV-1. HIV protease inhibitors include compounds having a peptidomimetic structure, high molecular weight (7600 Daltons) and substantial peptide character, e.g. CRIXIVAN (available from Merck) as well as nonpeptide protease inhibitors e.g., VIRACEPT (available from Agouron).

Viruses whose transmission may be inhibited by the antiviral activity of a combination therapy of this invention include, for example: human retroviruses, particularly HIV-1 and HIV-2 and the human T-lymphocyte viruses (HTLV-I and II); non-human retroviruses, including bovine leukosis virus, feline sarcoma and leukemia viruses, simian immunodeficiency, sarcoma and leukemia viruses, and sheep progress pneumonia viruses; non-retroviral viruses, including human respiratory syncytial virus, canine distemper virus, newcastle disease virus, human parainfluenza virus, influenza viruses, measles viruses, Epstein-Barr viruses, hepatitis B viruses, and simian Mason-Pfizer viruses; and non-enveloped viruses, including picomaviruses such as polio viruses, hepatitis A virus, enterovirus, echoviruses and coxsackie viruses, papovaviruses such as papilloma virus, parvoviruses, adenoviruses and reoviruses.

The present invention relates to compositions comprising a CCR5 antagonist and a CXCR4 antagonist. CXCR4 antagonists for use in the present disclosure include, but are not limited to, AMD070, AMD 3100, and AMD8664, all made by AnorMed, Inc., Langley, British Columbia, Canada, and CS-3995, and KRH-1120, KRH-2731, KRH-1636 made by Kureha Chemical Industry Co., Ltd., and Sankyo Co., Ltd., Japan. A discussion of the therapeutic potential of CXCR4 antagoinists in the treatment of HIV can be found in Expert Opinion on Investigational Drugs (2003) 12(2):185-195, and references disclosed therein. AMD070 can be given in single dose levels of 50, 100, 200, and 400 mg and multiple dose levels of 100 200, and 400 mg twice a day.

Compounds having the structural formulas I-V below, and pharmaceutically acceptable salts thereof, are collectively referred to herein as “CCR5 antagonists”. These compounds antagonize the CC chemokine receptor 5. Compounds of formula I and IlIl are described in U.S. Pat. Nos. 6,391,865, and 6,689,765. Compound of formula II and IV-V are described in U.S. Pat. Nos. 6,720,325; 7,060,701; and 7,098,213. Each of these U.S. patents are incorporated herein by reference in their entireties. In the compound of formula I:

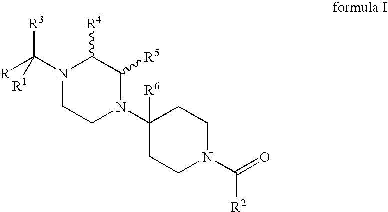

R can be R⁸-phenyl,—R⁸-pyridyl, R⁸-thiophenyl or -naphthyl;

R¹ can be hydrogen or C₁b—C₆ alkyl;

R² can be R⁹, R¹⁰, R¹¹-phenyl; R⁹, R¹⁰, R₁₁-substituted 6-membered heteroaryl; R⁹, R¹⁰, R¹¹-substituted 6-membered-heteroaryl N-oxide; R¹², R¹³ -substituted 5-membered heteroaryl; naphthyl; fluorenyl;

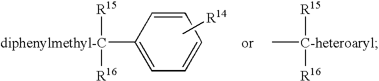

R³ can be hydrogen, C₁-C₆ alkyl, (C₁-C₆)alkoxy(C₁-C₆)alkyl, C₃-C₁₀ cycloalkyl, C₃-C₁₀cycloalkyl(C₁-C₆)alkyl, R⁸-phenyl, R⁸-phenyl(C₁-C₆)alkyl—R⁸-naphthyl, R⁸-naphthyl(C₁-C₆)alkyl, R⁸-heteroaryl or R⁸-heteroaryl(C₁—C₆)alkyl;

R⁴, R⁵, R⁷ and R¹³ can be independently selected from the group consisting of hydrogen and (C₁-C₆)alkyl;

R⁶ can be hydrogen, C₁-C₆ alkyl or C₂-C₆ alkenyl;

R⁸ can be 1 to 3 substituents independently selected from the group consisting of hydrogen, halogen, C₁-C₆ alkyl, C₁-C₆ alkoxy, —CF₃, CF₃O—, CH₃C(O)—, —CN, CH₃SO₂—, CF₃SO₂—, R¹⁴-phenyl, R¹⁴-benzyl, CH₃C(═NOCH₃), CH₃C(═NOCH₂CH₃), 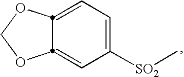
—NH₂, —NHCOCF₃, —NHCONH(C₁-C₆alkyl), —NHCO(C₁-C₆alkyl), —NHSO₂ (C₁-C₆alkyl), 5-membered heteroaryl and

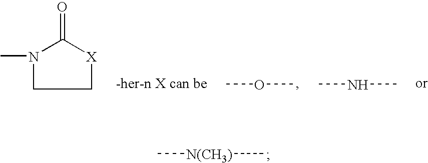

R⁹ and R¹⁰ can be independently selected from the group consisting of (C₁-C₆)alkyl, halogen, —NR¹⁷R¹⁸, —OH, —CF₃, —OCH₃, —O-acyl, —OCF₃ and —Si(CH₃)₃;

R¹¹ can be R⁹, hydrogen, phenyl, —NO₂, —CN, —CH₂F, —CHF₂, —CHO, —CH═NOR¹⁷, pyridyl, pyridyl N-oxide, pyrimidinyl, pyrazinyl, —N(R¹⁷)CONR¹⁸ R¹⁹, —NHCONH(chloro-(C₁-C₆)alkyl), —NHCONH((C₁-C₃)cycloalkyl(C₁C₆)alkyl), —NHCO(C₁-C₆)alkyl, —NHCOCF₃, —NHSO₂N((C₁-C₆)alkyl)₂, —NHSO₂(C₁-C₆)alkyl, —N(SO₂C—₃)₂, —NHCO₂(C₁-C₆)alkyl, C₃-C₁₀ cycloalkyl, —SR²⁰, —SOR²,—SO₂R²⁰, —SO₂NH(C₁-C₆alkyl), -□ydroxyl₁-C₆-alkyl, —OSO₂CF₃, hydroxy(C₁-C₆)alk-, —CONR¹⁷R¹⁸, —CON(—H₂CH₂ —O—CH₃)₂, —OCONH(C₁-C₆)a-yl, —CO₂R¹⁷, —Si(CH₃)₃ or —B —OC(CH₃)₂)₂;

R¹² can b -(C₁-C₆)alkyl, —NH₂ or R¹⁴-phenyl;

R¹⁴ can be 1 to 3 substituents independently selected from the group—consisting of hydrogen, (C₁-C₆)alky-, —CF₃, —CO₂R¹⁷, —CN, (C₁-C₆)alkoxy and halogen;

R¹⁵ and R¹⁶ can be independently selected from the group consisting of hydrogen and C₁-C₆ alkyl, or —¹⁵ and R¹⁶ together are a C₂-C₅ alkylene group and with the carbon to which they are attached form a spiro ring of 3 to 6 carbon atoms;

R¹⁷, R¹⁸ and R¹⁹ can be independently selected from the—group consisting of H and C₁-C₆ alkyl; and

R²⁰ can be C₁-C₆ alkyl or phenyl.

Non-limiting examples of compounds of formula I can be found in U.S. Pat. Nos. 6,391,865; 6,689,765; and 6,635, 646; and US published Appication Nos. 2004/0067961, 2004/0076609, and 2005/0065319, the disclosures of all of which are hereby incorporated by reference.

Certain compounds of the invention may exist in different isomeric forms (e.g., enantiomers, diastereoisomers, atropisomers and rotamers). The invention contemplates all such isomers both in pure form and in admixture, including racemic mixtures.

Certain compounds will be acidic in nature, e.g. those compounds which possess a carboxyl or phenolic hydroxyl group. These compounds may form pharmaceutically acceptable salts. Examples of such salts may include sodium, potassium, calcium, aluminum, gold and silver salts. Also contemplated are salts formed with pharmaceutically acceptable amines such as ammonia, alkyl amines, hydroxyalkylamines, N-methylglucamine and the like.

Certain basic compounds also form pharmaceutically acceptable salts, e.g., acid addition salts. For example, the pyrido-nitrogen atoms may form salts with strong acid, while compounds having basic substituents such as amino groups also form salts with weaker acids. Examples of suitable acids for salt formation are hydrochloric, sulfuric, phosphoric, acetic, citric, oxalic, malonic, salicylic, malic, fumaric, succinic, ascorbic, maleic, methanesulfonic and other mineral and carboxylic acids well known to those in the art. The salts are prepared by contacting the free base form with a sufficient amount of the desired acid to produce a salt in the conventional manner. The free base forms may be regenerated by treating the salt with a suitable dilute aqueous base solution such as dilute aqueous NaOH, potassium carbonate, ammonia and sodium bicarbonate. The free base forms differ from their respective salt forms somewhat in certain physical properties, such as solubility in polar solvents, but the acid and base salts are otherwise equivalent to their respective free base forms for purposes of the invention.

All such acid and base salts are intended to be pharmaceutically acceptable (i.e., non-toxic, physiologically acceptable) salts within the scope of the invention and all acid and base salts are considered equivalent to the free forms of the corresponding compounds for purposes of the invention.

Prodrugs and solvates of the compounds of the invention are also contemplated herein. A discussion of prodrugs is provided in T. Higuchi and V. Stella, Pro-rugs as Novel Delivery Systems (1987) 14 of the A. C. S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A. C. S. Symposium Series, and in Bioreversible Carriers in Drug Design, ed. Edward B. Roche, American Pharmaceutical Association and Pergamon Press, 1987.

For example, if a compound of Formula I or II or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C1-C2)alkyl and piperidino-, pyrrolidino- or morpholino(C₂-C₃)alkyl, and the like.

Similarly, if a compound of Formula I or II contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula I or II incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N- or di-N,N—(C₁-C₆)alkylaminoalkyl, —C(Y⁴)Y⁵ wherein Y⁴ is H or methyl and Y⁵ is mono-N- or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS Pharm Sci Tech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example 1. R. spectroscopy, show the presence of the solvent (or water) in the crystals as a solvate (or hydrate).

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

The present method of treating patients having HIV-1 infections comprises administering a therapeutically effective amount of a CXCR4 antagonist compound and a therapeutically effective amount of a CCR5 antagonist compound represented by structural formula I or II as a combination therapy or in association with a therapeutically effective amount of at least one of antiviral agent, alone or in combination with an anti-HIV-1 therapy, especially, HAART in accordance with good clinical practice to minimize HIV-1-RNA plasma levels. See for example A-M. Vandamme et al., in Antiviral Chemistry & Chemotherapy, 9:187-203 (1998) and “Drugs for HIV Infection” in The Medical Letter Vol. 39 (Issue 1015) Dec. 5, 1997, pages 111-116. In a preferred aspect of the present invention, the combination of a CXCR4 antagonist compound and a CCR5 antagonist of formulas I to II is administered to a patient infected with HIV-1, or co-infected with HIV-1 and HCV, optionally in association with ribavirin and HAART. It is a special feature of the present invention that each of a CXCR4 antagonist compound, the CCR5 antagonists of formulas I to II and optionally the components of HAART has a different mechanism of action in treating HIV-1. It is another special feature of the present invention that the CXCR4 antagonist compound and the CCR5 antagonists of formulas I to II are not expected to cause cross-resistance with each other or with the components of HAART. The initiation of the administration of a therapeutically effective amount of the combination of a CXCR4 antagonist compound, and a CCR5 antagonist compound represented by structural formula I or II and optionally HAART may occur before, after or concurrently with administering a therapeutically effective amount of a composition comprising a CXCR4 antagonist compound and a CCR5 antagonist compound represented by structural formula I or II in accordance with the present invention.

In an embodiment of the present invention, the method of treating patients having HIV-1 infections comprises two treatment time periods. In the first treatment time period, a combination of a therapeutically effective amount of a CXCR4 antagonist compound and a CCR5 antagonist compound represented by structural formula I or II is administered for a first treatment time period sufficient to lower HIV-1-RNA plasma levels, preferably by a power of 10, more preferably by at least two powers of ten, i.e., at least 10², lower than the initial HIV-1-RNA plasma level. In the second treatment time period, the method entails continuing the administration of a therapeutically effective amount of a combination of CXCR4 antagonist compound in association with a CCR5 antagonist compound represented by structural formula I or II and optionally a therapeutically effective amount of HAART in accordance with good clinical practice to minimize HIV-1-RNA plasma levels. A-M. Vandamme et al., Antiviral Chemistry & Chemotherapy, 9:187-203 (1998) disclose current clinical treatments of HIV-1 infections, including when to start multidrug therapy and which drugs to combine. The triple drug therapy may include two NRTIs and one PI, but there are many issues to be considered in the choice of the precise HAART for any patient. See for example, Tables 1 & 2 and FIG. 2in A-M. Vandamme et al., listed hereinabove.

One or more, preferably one to four, antiviral agents useful in anti-HIV-1 therapy may be used in combination with a CXCR4 antagonist compound and a CCR5 antagonist of the present invention. The antiviral agent or agents may be combined with the CXCR4 antagonist compound and CCR5 antagonist in a single dosage form, or the CXCR4 antagonist compound and CCR5 antagonist and the antiviral agent or agents may be administered simultaneously or sequentially as separate dosage forms. The antiviral agents contemplated for use in combination with the compounds of the present invention comprise nucleoside and nucleotide reverse transcriptase inhibitors, non-nucleoside reverse transcriptase inhibitors, protease inhibitors and other antiviral drugs. Moreover, antiviral agents not falling within these classifications are also contemplated. In particular, the combinations known as HAART (Highly Active Antiretroviral Therapy) are contemplated for use in combination with the composition of this invention.

Typical suitable NRTIs include zidovudine (AZT) available under the RETROVIR tradename from Glaxo-Wellcome Inc., Research Triangle, N.C. 27709; didanosine (ddl) available under the VIDEX tradename from Bristol-Myers Squibb Co., Princeton, N.J. 08543; stavudine (d4T) available under the ZERIT trademark from Bristol-Myers Squibb Co., Princeton, N.J. 08543; lamivudine (3TC) available under the EPIVIR tradename from Glaxo-Wellcome Research Triangle, N.C. 27709; abacavir (1592U89) disclosed in WO96/30025 and available under the ZIAGEN trademark from Glaxo-Wellcome Research Triangle, N.C. 27709; adefovir dipivoxil [bis(POM)-PMEA] available under the PREVON tradename from Gilead Sciences, Foster City, Calif. 94404; BCH-10652, a reverse transcriptase inhibitor (in the form of a racemic mixture of BCH-10618 and BCH-10619) under development by Biochem Pharma, Laval, Quebec H7V, 4A7, Canada; EMTRIVA® from Gilead Sciences, emitricitabine [(−)-FTC] licensed from Emory University under Emory Univ. U.S. Pat. No. 5,814,639 and under development by Triangle Pharmaceuticals, Durham, N.C. 27707 (now Gilead Sciences); TENOFOVIR, (bis-(POM).PMPA, Gilead Sciences; beta-L-FD4 (also called‘bet’-L-D4C and named beta-L-2′, 3′-dideoxy-5-fluoro-cytidene) licensed by Yale University to Achillion Pharmaceuticals, New Haven Conn. 06511; DAPD, the purine nucleoside, (−)beta-D-2,6,-diamino-purine dioxolane disclosed in EP 0656778 and licensed by Emory University and the University of Georgia to Triangle Pharmaceuticals, Durham, N.C. 27707; lodenosine (FddA), 9-(2,3-dideoxy-2-fluoro-b-D-threo-pentofuranosyl)adenine, a acid stable purine-based reverse transcriptase inhibitor discovered by the NIH and under development by U.S. Bioscience Inc., West Conshohoken, Pa. 19428; and Reverset, licensed by Emory University to Pharmasset and subsequently to InCyte Pharmaceuticals, Princeton, N.J.

Typical suitable NNRTIs include nevirapine (BI-RG-587) available under the VIRAMUNE tradename from Boehringer Ingelheim, the manufacturer for Roxane Laboratories, Columbus, Ohio 43216; etravirine (TMC-125; available from Tibotec); delaviradine (BHAP, U-90152) available under the RESCRIPTOR tradename from Pharmacia & Upjohn Co., Bridgewater N.J. 08807; efavirenz (DMP-266) a benzoxazin-2-one disclosed in WO94/03440 and available under the SUSTIVA tradename from DuPont Pharmaceutical Co., Wilmington, Del. 19880-0723; PNU-142721, a furopyridine-thio-pyrimide under development by Pharmacia and Upjohn, Bridgewater N.J. 08807; AG-1549 (formerly Shionogi #S-1153); 5-(3, 5-dichlorophenyl)- thio-4-isopropyl-1-(4-pyridyl)methyl-1H-imidazol-2-ylmethyl carbonate disclosed in WO 96/10019 and under clinical development by Agouron Pharmaceuticals, Inc., LaJolla Calif. 92037-1020; MKC-442 (1-(ethoxy-methyl)-5-(1-methylethyl)-6-(phenylmethyl)-(2,4(1H ,3H)-pyrimidi nedione) discovered by Mitsubishi Chemical Co. and under development by Triangle Pharmaceuticals, Durham, N.C. 27707; and (+)-calanolide A (NSC-675451) and B, coumarin derivatives disclosed in NIH U.S. Pat. No. 5,489,697, licensed to Med Chem Research, which is co-developing (+) calanolide A with Vita-invest as an orally administrable product.

Typical suitable Pls include saquinavir (Ro 31-8959) available in hard gel capsules under the INVIRASE tradename and as soft gel capsules under the FORTOVASE tradename from Roche Pharmaceuticals, Nutley, N.J. 07110-1199; ritonavir (ABT-538) available under the NORVIR tradename from Abbott Laboratories, Abbott Park, Ill. 60064; indinavir (MK-639) available under the CRIXIVAN tradename from Merck & Co., Inc., West Point, Pa. 19486-0004; nelfnavir (AG-1343) available under the VIRACEPT tradename from Agouron Pharmaceuticals, Inc., LaJolla Calif. 92037-1020; amprenavir (141W94), tradename AGENERASE, a non-peptide protease inhibitor under development by Vertex Pharmaceuticals, Inc., Cambridge, Mass. 02139-4211 and available from Glaxo-Wellcome, Research Triangle, N.C. under an expanded access program; ATAZANAVIR available from Bristol-Myers Squibb, Princeton, N.J. 08543 (originally discovered by Novartis, Basel, Switzerland (CGP-61755); DMP-450, a cyclic urea discovered by Dupont and under development by Triangle Pharmaceuticals; BMS-232632, an azapeptide under development by Bristol-Myers Squibb, Princeton, N.J. 08543, as a 2nd-generation HIV-1 PI; ABT-378 under development by Abbott, Abbott Park, Ill. 60064; AG-1549 an orally active imidazole carbamate discovered by Shionogi (Shionogi #S-1153) and under development by Agouron Pharmaceuticals, Inc., LaJolla Calif. 92037-1020; TMC-114, Tibotec, subsidiary of Johnson & Johnson; and TIPRANAVIR® made by Boeringer Engelheim, Ridgefield, Conn.

Other antiviral agents include hydroxyurea, ribavirin, IL-2, IL-12, pentafuside and Yissum Project No.11607. Hydroxyurea (Droxia), a ribonucleoside triphosphate reductase inhibitor, the enzyme involved in the activation of T-cells, was discovered at the NCI is under development by Bristol-Myers Squibb; in preclinical studies, it was shown to have a synergistic effect on the activity of didanosine and has been studied with stavudine. IL-2 is disclosed in Ajinomoto EP-0142268, Takeda EP-0176299, and Chiron U.S. Pat. Nos. RE 33,653, 4,530,787, 4,569,790, 4,604,377, 4,748,234, 4,752,585, and 4,949,314 is available under the PROLEUKIN (aldesleukin) tradename from Chiron Corp., Emeryville, Calif. 94608-2997 as a lyophilized powder for IV infusion or sc administration upon reconstitution and dilution with water; a dose of about 1 to about 20 million IU/day, sc is preferred; a dose of about 15 million IU/day, sc is more preferred. IL-12 is disclosed in WO96/25171 and is available from Roche Pharmaceuticals, Nutley, N.J. 07110-1199 and American Home Products, Madison, N.J. 07940; a dose of about 0.5 microgram/kg/day to about 10 microgram/kg/day, sc is preferred. Pentafuside FUZEON® of Trimeris and Roche (DP-178, T-20) a 36-amino acid synthetic peptide, disclosed in U.S. Pat. No. 5,464,933 licensed from Duke University to Trimeris. Enfuvirtide acts by inhibiting fusion of HIV-1 to target membranes. Enfuvirtide (3-100 mg/day) is given as a continuous sc infusion or injection to'ether with efavirenz and 2 PI's to HIV-1 positive patients refractory to a triple combination therapy; use of 100 mg/day is preferred. BMS-806 is an entry inhibitor under development by BMS. Other inhibitors under development include integrase inhibitors b - Merck & Co. Ribavirin, 1-β-D-ribofuranosyl-1H-1,2,4-triazole-3-carboxamide, is available from ICN Pharmaceuticals, Inc., Costa Mesa, Calif; its manufacture and formulation are described in U.S. Pat. No. 4,211,771.

The term “anti-HIV-1 therapy” as used herein means any anti-HIV-1 drug found useful for treating HIV-1 infections in man alone, or as part of multidrug combination therapies, especially the HAART triple and quadruple combination therapies. Typical suitable known anti-HIV-1 therapies include, but are not limited to multidrug combination therapies such as (i) at least three anti-HIV-1 drugs selected from two NRTIs, one PI, a second PI, and one NNRTI; and (ii) at least two anti-HIV-1 drugs selected from, NNRTIs a—PIs. Typical suitable HAART—multidrug combination therapies include: (a) triple combination therapies such as two NRTIs and one PI; or (b) two NRTIs and one NNRTI; and (c) quadruple combination therapies such as two NRTIs , one PI and a second PI ol NNRTI. In treatment of naive patients, it is preferred to start anti-HIV-1 treatment with the triple combination therapy; the use of two NRTIs and one PI is preferred unless there is intolerance to PIs. Drug compliance is essential. The CD4⁺and HIV-1-RNA plasma levels should be monitored every 3-6 months. Should viral load plateau, a fourth drug, e.g., one PI or one NNRTI could be added. See the table below wherein non-limiting examples of typical therapies are further described. The present invention also contemplates individualized treatment therapies.

### ANTI-HIV-1 Multi Drug Combination Therapies

A. Triple Combination Therapies
1. Two NRTIs¹+one PI²
2. Two NRTIs¹+one NNRTI³

B. Quadruple Combination Therapies⁴

1. Two NRTle PI+a second PI or one NNRTI

C. Alternatives⁵
Two NRTI¹
One NRTI⁵+one pl²
Two PIs⁶+one NRTI⁷ or NNRTI³
One pI²+one NRTone NNRTI³

Footnotes to Table
1. 1. One of the following: zidovudine+lamivudine; zidovudine+didanosine; stavudine+lamivudine; stavudine+didanosine; zidovudine+zalcitabine
   2. Indinavir, nelfinavir, ritonavir or saquinavir soft gel capsules.
   3. Nevirapine or delavirdine.
   4. See A-M. Vandamne et al Antiviral Chemistry & Chemotherapy 9:187 at p 193-197 and FIGS. 30 2+L.
   5. Alternative regimens are for patients unable to take a recommended regimen because of compliance problems or toxicity, and for those who fail or relapse on a recommended regimen. Double nucleoside combinations may lead to HIV-resistance and clinical failure in many patients.
   6. Most data obtained with saquinavir and ritonavir (each 400 mg bid).
   7. Zidovudine, stavudine or didanosine.

Agents known in the treatment of rheumatoid arthritis, transplant and graft v. host disease, inflammatory bowel disease and multiple sclerosis which can be administered in combination with the disclosed composition are as follows: solid organ transplant rejection and graft v. host disease: immune suppressants such as cyclosporine and Interleukin-10 (IL-10), tacrolimus, antilymphocyte globulin, OKT-3 antibody, and steroids; inflammatory bowel disease: IL-10 (see U.S. Pat. No. 5,368,854), steroids and azulfidine; rheumatoid arthritis: methotrexate, azathioprine, cyclophosphamide, steroids and mycophenolate mofetil; multiple sclerosis: interferon-beta, interferon-alpha, and steroids.

For preparing pharmaceutical compositions of the CXCR4 antagonist compound and CCR5 antagonist compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be fund in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compositions of the invention may also be deliverable transdermally. The transdermal compositions can take the form of creams, lotions, aerosols and/or emulsions and can be included in a transdermal patch of the matrix or reservoir type as are conventional in the art for this purpose.

Preferably the composition is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 10 mg to about 500 mg, preferably from about 25 mg to about 300 mg, more preferably from about 50 mg to about 250 mg, and most preferably from about 55 mg to about 200 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the composition and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 100 mg/day to about 300 mg/day, preferably 150 mg/day to 250 mg/day, more preferably about 200 mg/day, in two to four divided doses.

The doses and dosage regimen of the NRTIs, NNRTIs, Pis and other agents will be determined by attending clinician in view of the approved doses and dosage regimen in the package insert or as set forth in the protocol taking into consideration the age, sex and condition of the patient and the severity of the HIV-1 infection.

A person suffering from chronic hepatitis C infection may exhibit one or more of the following signs or symptoms:
(a) elevated ALT,
(b) positive test for anti-HCV antibodies,
(c) presence of HCV as demonstrated by a positive test for the presence of HCV-RNA in the serum,
(d) clinical stigmata of chronic liver disease,
(e) hepatocelluar damage.

In a preferred aspect of the present invention, a therapeutically effective amount of the combination therapy of a CXCR4 antagonist compound and a CCR5 antagonist compound represented by structural formula I or II is administered optionally in association with a therapeutically effective amount of an antiviral agent, e.g., ribavirin, and anti-retroviral therapy, e.g., HMRT, to the patient having HIV-1 infection and exhibiting one or more of the above signs or symptoms in the first and second treatment time periods in amounts sufficient to eliminate or at least alleviate one or more of the signs or symptoms, and to lower the HCV-RNA plasma levels by at least a power of ten, and preferably to eradicate detectable HCV-RNA at least by the end of the second treatment time period and to maintain no detectable HCV-RNA for at least 24 weeks after the end of the second treatment time period. The sum of the first and second treatment time periods is about 40-50 weeks, and preferably is 48 weeks. Administration of the antiviral agent may be discontinued after the end of the second time period depending upon the judgment of the attending clinician.

The term “no detectable HCV-RNA” in the context of the present invention means that there are fewer than 100 copies of HCV-RNA per ml of plasma of the patient as measured by quantitative, multi-cycle reverse transcriptase PCR methodology. HCV-RNA is preferably measured in the present invention by research-based RT-PCR methodology well known to the skilled clinician. This methodology is referred to herein as HCV-RNA/qPCR. The lower limit of detection of HCV-RNA is 100 copies/mL. Serum HCV-RNA/qPCR testing and HCV genotype testing will be performed by a central laboratory. See also J. G. McHutchinson et al. (N. Engl. J. Med., 1998, 339:1485-1492), and G. L. Davis et al. (N. Engl. J. Med. 339:1493-1499).

In a preferred embodiment of the present invention, those patients co-infected with HIV-1 and HCV infections are treated with a combination therapy of a CXCR4 antagonist compound and a CCR5 antagonist compound represented by structural formula I or II optionally in association with an antiviral agent and a HAART combination considered appropriate by the attending clinician and the patient. Ribavirin, 1-β-D-ribofuranosyl-1 H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771.

For the pediatric patient co-infected with the HIV-1 and HCV infections, a suitable HMRT includes a NRTI+a PI, e.g., Nelfinavir+a NNRTI, e.g., Efavirenz in combination with the dosages and dosage regimens for a CXCR4 antagonist compound and a CCR5 antagonist compound listed herein above. A human growth hormone such as the polypeptide hormone, somatropin, of recombinant rDNA origin, available under the HUMATROPE tradename from Eli Lilly & Co., Indianapolis, Ind. 46285, may be administered to these pediatric patients in the dosage and administration schedule listed in the product information sheet in consultation with the attending clinician to reduce retardation of growth.

HAART is optionally administered to the patient in association with a CXCR4 antagonist compound and a CCR5 antagonist compound, that is, the CXCR4 antagonist compound and a CCR5 antagonist compound dose may be administered before, after or during the same period of time that the patient receives doses of HAART.

In a preferred embodiment of the present invention, the disclosed composition is administered to HIV-1 infected patients prior to initiation of HAART, and preferably about two to about four weeks prior to initiation of HAART. In another preferred embodiment of the present invention, administration of a CXCR4 antagonist compound is initiated concurrently, i.e., on the same day with the administration of a CCR5 antagonist compound represented by structural formula I or II and optionally HAART. In another preferred embodiment of the present invention the CXCR4 antagonist compound is administered after the HIV-1 infected patient has initiated use of a CCR5 antagonist compound represented by structural formula I or II and optionally HAART.

The goal of the HIV-1 therapy of the present invention is to reduce the HIV-1-RNA viral load below the detectable limit. The “detectable limit of HIV-1-RNA” in the context of the present invention means that there are fewer than about 200 to fewer than about 50 copies of HIV-1-RNA per ml of plasma of the patient as measured by quantitative, multi-cycle reverse transcriptase PCR methodology. HIV-1-RNA is preferably measured in the present invention by the methodology of Amplicor-1 Monitor 1.5 (available from Roche Diagnostics)or of Nuclisens HIV-1 QT-1. This methodology is described by Schooley, R T, Antiviral Therapy(1997), 2 (Suppl. 4):59-70.

The doses and dosage regimen of the NRTIs, NNRTIs, PI, enfuvirtide, IL-2, IL-12, a CCR5 antagonist compound represented by structural formula I or II and a CXCR4 antagonist compound will be determined by attending clinician in view of the approved doses and dosage regimen in the package insert or as set forth in the protocol taking into consideration the age, sex and condition of the patient and the severity of the HIV-1 and HCV infections. For the pediatric patient infected with the HIV-1, or co-infected with the HIV-1 and HCV infections a suitable HAART includes a NRTI+a PVI, e.g., Nelfinavir+a NNRTI, e.g., Efavirenz in combination with the dosages and dosage regimens for CXCR4 antagonist compound and a CCR5 antagonist compound listed herein above.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that can vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” include plural referents unless expressly and unequivocally limited to one referent. Thus, for example, reference to “a carrier” includes two or more different carriers. As used herein, the term “include” and its grammatical variants are intended to be non-limiting, such that recitation of items in a list is not to the exclusion of other like items that can be substituted or added to the listed items.

While particular embodiments have been described, alternatives, modifications, variations, improvements, and substantial equivalents that are or can be presently unforeseen can arise to applicants or others skilled in the art. Accordingly, the appended claims as filed and as they can be amended are intended to embrace all such altematives, modifications variations, improvements, and substantial equivalents.

What is claimed is:

1. A composition comprising a CXCR4 antagonist and a CCR5 antagonist represented by formula I: 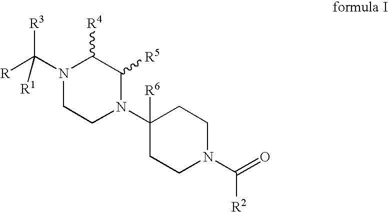
or a pharmaceutically acceptable salt or solvate thereof,
wherein R is optionally substituted phenyl, pyridyl, thiophenyl or naphthyl;
R¹ is hydrogen or alkyl;
R² is substituted phenyl, substituted heteroaryl, naphthyl, fluorenyl, diphenylmethyl or optionally substituted phenyl- or heteroaryl-alkyl;
R³ is hydrogen, alkyl, alkoxyalkyl, cycloalkyl, cycloalkylalkyl, or optionally substituted phenyl, phenylalkyl, naphthyl, naphthylalkyl, heteroaryl or heteroarylalkyl;
R⁴, R⁵ and R⁷ are hydrogen or alkyl; and
R⁶ is hydrogen, alkyl or alkenyl.

2. A composition comprising a CXCR4 antagonist and a CCR5 antagonist represented by formula II: 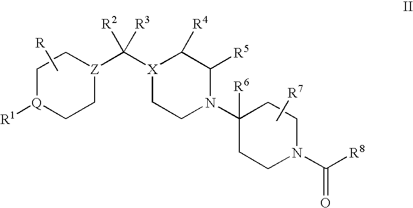
or a pharmaceutically acceptable salt, solvate, or ester thereof, wherein:
Q, X and Z are independently selected from the group consisting of CH and N, provided that one or both of Q and Z is N;
R, R⁴, R⁵, R⁶ and R⁷ are independently selected from the group consisting of H and (C₁-C₆)alkyl;
R¹ is H, (C₁-C₆)alkyl, fluoro-(C₁-C₆)alkyl-, R⁹-aryl(C₁-C₆)alkyl-, R⁹-heteroaryl-(C₁-C₆)alkyl-, (C₁-C₆)alkyl-SO₂—, (C₃-C₆)cycloalkyl-SO₂—, fluoro-(C₁-C₆)alkyl-SO₂—, R⁹-aryl-SO₂—, R⁹-heteroaryl-SO₂—, N(R²²)(R²³)—SO₂—, (C₁-C₆)alkyl-C(O)—, (C₃-C₆)cyclo-alkyl-C(O)—, fluoro-(C₁-C₆)alkyl-C(O)—, R⁹-aryl-C(O)—, NH—(C₁-C₆)alkyl-C(O)— or R⁹-aryl-NH—C(O)—;
R² is H or (C₁-C₆)alkyl, and R³ is H, (C₁-C₆)alkyl, (C₁-C₆)alkoxy(C₁-C₆)alkyl-, (C₃-C₁₀)-cycloalkyl-, (C₃-C₁₀)cycloalkyl(C₁-C₆)alkyl-, R⁹-aryl, R⁹-aryl(C₁-C₆)-alkyl-, R⁹-heteroaryl, or R⁹-heteroaryl(C₁-C₆)alkyl-, provided that both X and Z are not each N;
or R² and R³ together are ═O, ═NOR¹⁰, ═N—NR¹¹R¹² or ═CH(C₁-C₆)alkyl, provided that when one or both of X and Z is N, R² and R³ together are not ═CH(C₁-C₆)alkyl;
and when X and Z are each CH, R³ can also be (C₁-C₆)alkoxy, R⁹-aryloxy, R⁹-heteroaryloxy, (C₁-C₆)alkyl-C(O)O—, (C₁-C₆)alkyl-NH—C(O)O—, N((C₁-C₆)alkyl)₂—C(O)O—, (C₁-C₆)alkyl-C(O)—NR¹³—, (C₁-C₆)alkyl-O—C(O)—NR¹³—, (C₁-C₆)alkyl-NH—C(O)—NR¹³— or N((C₁-C₆)alkyl)₂—C(O)—NR¹³—;
R⁸ is (R¹⁴,R¹⁵,R¹⁶)-substituted phenyl, (R¹⁴,R¹⁵,R¹⁶)-substituted 6-membered heteroaryl, (R¹⁴,R⁵¹,R¹⁶)-substituted 6-membered heteroaryl N-oxide, (R¹⁷,R¹⁸)-substituted 5-membered heteroaryl, naphthyl, fluorenyl, diphenylmethyl, 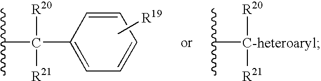
R⁹ is 1, 2 or 3 substituents independently selected from the group consisting of H, halogen, (C₁-C₆)alkyl, (C₁-C₆)alkoxy, —CF₃, —OCF₃, CH₃C(O)—, —CN, CH₃SO₂—, CF₃SO₂— and —N(R²²)(R²³);
R¹⁰ is H, (C₁-C₆)alkyl, fluoro(C₁-C₆)alkyl-□ydroxyl₁₀)cycloalkyl(C₁-C₆)alkyl-, hydroxy(C₂-C₆)alkyl-, (C₁-C₆)alkyl-O—(C₂-C₆)alkyl-, (C₁-C₆)alkyl-O—C(O)—(C₁-C₆)alkyl- or N(R²²)(R²³)—C(O)—(C₁-C₆)alkyl-;
R¹¹ and R¹² are independently selected from the group consisting of H, (C₁-C₆)alkyl and (C₃-C₁₀)cycloalkyl, or R¹¹ and R¹² together are C₂-C₆ alkylene and form a ring with the nitrogen to which they are attached;
R¹⁴ and R¹⁵ are independently selected from the group consisting of (C₁-C₆)alkyl, halogen, —NR²²R²³, —OH, —CF₃, —OCH₃, —O-acyl and —OCF₃;
R¹⁶ is R¹⁴, hydrogen, phenyl, —NO₂, —CN, —CH₂F, —CHF₂, —CHO, —CH═NOR²⁴, pyridyl, pyridyl N-oxide, pyrimidinyl, pyrazinyl, —N(R²⁴)CONR²⁵R²⁶, —NHCONH(chloro-(C₁-C₆)alkyl), —NHCONH((C₃-C₁₀)cycloalkyl(C₁-C₆)alkyl), —NHCO(C₁-C₆)alkyl, —NHCOCF₃, —NHSO₂N(R²²)(R²³), —NHSO₂(C₁-C₆)alkyl, —N(SO₂CF₃)₂, —NHCO₂—(C₁-C₆)alkyl, C₃-C₁₀ cycloalkyl, —SR²⁷, —SOR²⁷, —SO₂R²⁷, —SO₂NH(R^{□ydroxyl}OSO₂(C₁-C₆)alkyl, —OSO₂CF₃, hydroxy(C₁-C₆)alkyl-, —CON R²⁴R²⁵, —CON(CH₂CH₂OCH₃)₂, —OCONH(C₁-C₆)alkyl, —CO₂R²⁴, —Si(CH₃)₃ or —B(OC(CH₃)₂)₂;
R¹⁷ is (C₁-C₆)alkyl, —N(R²²)(R²³) or R¹⁹-phenyl;
R¹³, R¹⁸, R²², R²³, R²⁴, R²⁵ and R²⁶ are independently selected from the group consisting of H and (C₁-C₆)alkyl;
R¹⁹ is 1, 2 or 3 substituents independently selected from the group consisting of H, (C₁-C₆)alkyl, —CF₃, —CO₂R²⁵, —CN, (C₁-C₆)alkoxy and halogen;
R²⁰ and R²¹ are independently selected from the group consisting of H and (C₁-C₆)alkyl, or R²⁰ and R²¹ together with the carbon to which they are attached form a spiro ring of 3 to 6 carbon atoms; and
R²⁷ is (C₁-C₆)alkyl or phenyl.

3. The composition of claim 1, wherein the CXCR4 antagonist is at least one of AMD-070, CS-3955, KRH-1120, KRH-2731, and KRH-1636.

4. The composition of claim 3, wherein the CXCR4 antagonist is CS-3955.

5. The composition of claim 3, wherein the CXCR4 antagonist is AMD-070.

6. The composition of claim 3, wherein the CXCR4 antagonist is at least one of KRH-1120, KRH-2731, and KRH-1636.

7. The composition of claim 2, wherein the CXCR4 antagonist is at least one of AMD-070, CS-3955, KRH-1120, KRH-2731, and KRH-1636.

8. The composition of claim 7, wherein the CXCR4 antagonist is CS-3955.

9. The composition of claim 7, wherein the CXCR4 antagonist is AMD-070.

10. The composition of claim 7, wherein the CXCR4 antagonist is at least one of KRH-1120, KRH-2731, and KRH-1636.

11. The composition of claim 1, wherein the CCR5 antagonist of formula I is a compound of formula III: 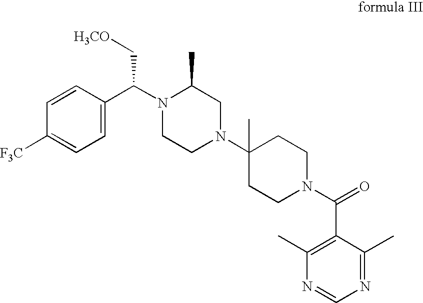
or a pharmaceutically acceptable salt or solvate thereof.

12. The composition of claim 2, wherein the CCR5 antagonist of formula I is a compound of formula IV: 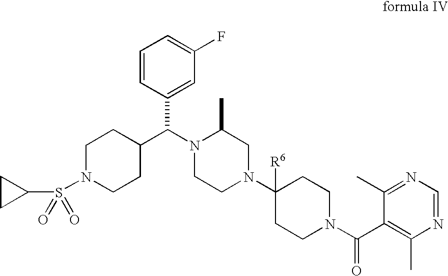
or a pharmaceutically acceptable salt or solvate thereof.

13. The composition of claim 2, wherein the CCR5 antagonist of formula I is a compound of formula V: 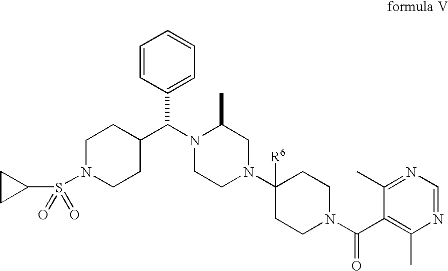
or a pharmaceutically acceptable salt of solvate thereof.

14. A composition comprising a CXCR4 antagonist and a CCR5 antagonist of formula III: 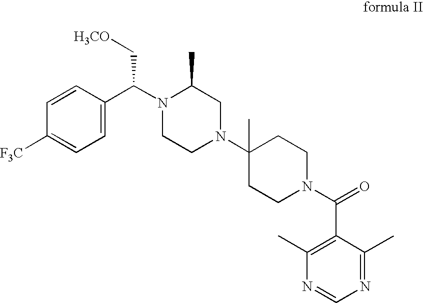
or a pharmaceutically acceptable salt or solvate thereof.

15. The composition of claim 14, wherein the CXCR4 antagonist is at least one of AMD-070, CS-3955, KRH-1120, KRH-2731, and KRH-1636.

16. The composition of claim 15, wherein the CXCR4 antagonist is CS-3955.

17. The composition of claim 15, wherein the CXCR4 antagonist is AMD-070.

18. The composition of claim 15, wherein the CXCR4 antagonist is at least one of KRH-1120, KRH-2731, KRH-1636.

19. A pharmaceutical composition comprising a CCR5 antagonist, a CXCR4 antagonist, and a pharmaceutically effective carrier.

20. The pharmaceutical composition of claim 19, wherein the CCR5 antagonist is represented by formula I: 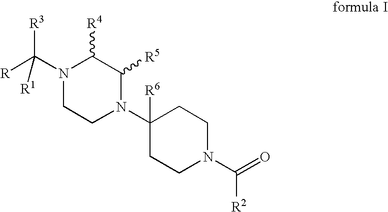
or a pharmaceutically acceptable salt or solvate thereof,
wherein R is optionally substituted phenyl, pyridyl, thiophenyl or naphthyl;
R¹ is hydrogen or alkyl;
R² is substituted phenyl, substituted heteroaryl, naphthyl, fluorenyl, diphenylmethyl or optionally substituted phenyl- or heteroaryl-alkyl;
R³ is hydrogen, alkyl, alkoxyalkyl, cycloalkyl, cycloalkylalkyl, or optionally substituted phenyl, phenylalkyl, naphthyl, naphthylalkyl, heteroaryl or heteroarylalkyl;
R⁴, R⁵ and R⁷ are hydrogen or alkyl; and
R⁶ is hydrogen, alkyl or alkenyl.

21. The pharmaceutical composition of claim 19, wherein the CCR5 antagonist is represented by formula II: 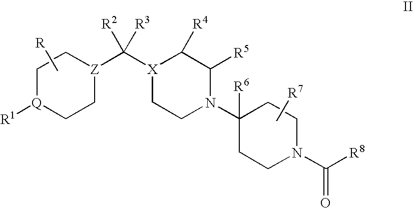
or a pharmaceutically acceptable salt, solvate, or ester thereof, wherein:
Q, X and Z are independently selected from the group consisting of CH and N, provided that one or both of Q and Z is N;
R, R⁴, R⁵, R⁶ and R⁷ are independently selected from the group consisting of H and (C₁-C₆)alkyl;
R¹ is H, (C₁-C₆)alkyl, fluoro-(C₁-C₆)alkyl-, R⁹-aryl(C₁-C₆)alkyl-, R⁹-heteroaryl-(C₁-C₆)alkyl-, (C₁-C₆)alkyl-SO₂—, (C₃-C₆)cycloalkyl-SO₂—, fluoro-(C₁-C₆)alkyl-SO₂—, R⁹-aryl-SO₂—, R⁹-heteroaryl-SO₂—, N(R²²)(R²³)—SO₂—, (C₁-C₆)alkyl—C(O)—, (C₃-C₆)cyclo-alkyl-C(O)—, fluoro-(C₁-C₆)alkyl-C(O)—, R⁹-aryl-C(O)—, NH—(C₁-C₆)alkyl-C(O)— or R⁹-aryl-NH—C(O)—;
R² is H or (C₁-C₆)alkyl, and R³ is H, (C₁-C₆)alkyl, (C₁-C₆)alkoxy(C₁-C₆)alkyl-, (C₃-C₁₀)-cycloalkyl-, (C₃-C₁₀)cycloalkyl(C₁-C₆)alkyl-, R⁹-aryl, R⁹-aryl(C₁-C₆)-alkyl-, R⁹-heteroaryl, or R⁹-heteroaryl(C₁-C₆)alkyl-, provided that both X and Z are not each N;
or R² and R³ together are ═O, ═NOR¹⁰, ═N—NR¹¹R¹² or ═CH(C₁-C6)alkyl, provided that when one or both of X and Z is N, R² and R³ together are not ═CH(C₁-C₆)alkyl;
and when X and Z are each CH, R³ can also be (C₁-C₆)alkoxy, R⁹-aryloxy, R⁹-heteroaryloxy, (C₁-C₆)alkyl-C(O)O—, (C₁-C₆)alkyl-NH—C(O)O—, N((C₁-C₆)alkyl)₂—C(O)O—, (C₁-C₆)alkyl-C(O)—NR¹³—, (C₁-C₆)alkyl-O—C(O)—NR¹³—, (C₁-C₆)alkyl-NH—C(O)—NR¹³— or N((C₁-C₆)alkyl)₂—C(O)—NR¹³—;
R⁸ is (R¹⁴,R ¹⁵,R¹⁶)-substituted phenyl, (R¹⁴,R¹⁵,R¹⁶)-substituted 6-membered heteroaryl, (R¹⁴, R¹⁵, R¹⁶)-substituted 6-membered heteroaryl N-oxide, (R¹⁷,R¹⁸)-substituted 5-membered heteroaryl, naphthyl, fluorenyl, 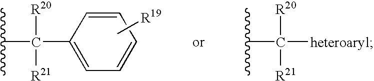
diphenylmethyl,
R⁹ is 1, 2 or 3 substituents independently selected from the group consisting of H, halogen, (C₁-C₆)alkyl, (C₁-C₆)alkoxy, —CF₃, —OCF₃, CH₃C(O)—, —CN, CH₃SO₂—, CF₃SO₂— and —N(R²²)(R²³);
R¹⁰ is H, (C₁-C₆)alkyl, fluoro(C₁-C₆)alkyl-, (C₃-C₁₀)cycloalkyl(C₁-C₆)alkyl-, hydroxy(C₂-C₆)alkyl-, (C₁-C₆)alkyl-O—(C₂-C₆)alkyl-, (C₁-C₆)alkyl-O—C(O)—(C₁-C₆)alkyl- or N(R²²)(R²³)—C(O)—(C₁-C₆)alkyl-;
R¹¹ and R¹² are independently selected from the group consisting of H, (C₁-C₆)alkyl and (C₃-C₁₀)cycloalkyl, or R¹¹ and R¹² together are C₂-C₆ alkylene and form a ring with the nitrogen to which they are attached;
R¹⁴ and R¹⁵ are independently selected from the group consisting of (C₁-C₆)alkyl, halogen, —NR²²R²³, —OH, —CF₃, —OCH₃, —O-acyl and —OCF₃;
R¹⁶ is R¹⁴, hydrogen, phenyl, —NO₂, —CN, —CH₂F, —CHF₂, —CHO, —CH═NOR²⁴, pyridyl, pyridyl N-oxide, pyrimidinyl, pyrazinyl, —N(R²⁴)CONR²⁵R²⁶, —NHCONH(chloro-(C₁-C₆)alkyl), —NHCONH((C₃-C₁₀)cycloalkyl(C₁-C₆)alkyl), —NHCO(C₁-C₆)alkyl, —NHCOCF₃, —NHSO₂N(R²²)(R²³), —NHSO₂(C₁-C₆)alkyl, —N(SO₂CF₃)₂, —NHCO₂-(C₁-C₆)alkyl, C₃-C₁₀ cycloalkyl, —SR²⁷, —SOR²⁷, —SO₂R²⁷, —SO₂NH(R²²), —OSO₂(C₁-C₆)alkyl, —OSO₂CF₃, hydroxy(C₁-C₆)alkyl-, —CON R²⁴R²⁵, —CON(CH₂CH₂OCH₃)₂, —OCONH(C₁-C₆)alkyl, —CO₂R²⁴, —Si(CH₃)₃ or —B(OC(CH₃)₂)₂;
R¹⁷ is (C₁-C₆)alkyl, —N(R²²)(R²³) or R¹⁹-phenyl;
R¹³, R¹⁸, R²², R²³, R²⁴, R²⁵ and R²⁶ are independently selected from the group consisting of H and (C₁-C₆)alkyl;
R¹⁹ is 1, 2 or 3 substituents independently selected from the group consisting of H, (C₁-C₆)alkyl, —CF₃, —CO₂R²⁵, —CN, (C₁-C₆)alkoxy and halogen;
R²⁰ and R²¹ are independently selected from the group consisting of H and (C₁-C₆)alkyl, or R²⁰ and R²¹ together with the carbon to which they are attached form a spiro ring of 3 to 6 carbon atoms; and
R²⁷ is (C₁-C₆)alkyl or phenyl.

22. The pharmaceutical composition of claim 20, wherein the CCR5 antagonist of formula I is a compound of formula III: 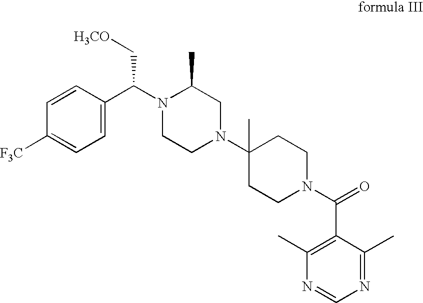
or a pharmaceutically acceptable salt or solvate thereof.

23. The pharmaceutical composition of claim 21, wherein the CCR5 antagonist of formula II is a compound of formula IV: 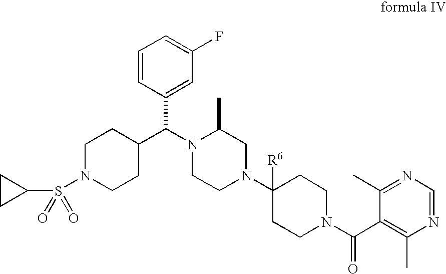
or a pharmaceutically acceptable salt or solvate thereof.

24. The pharmaceutical composition of claim 21, wherein the CCR5 antagonist of formula II is a compound of formula V: 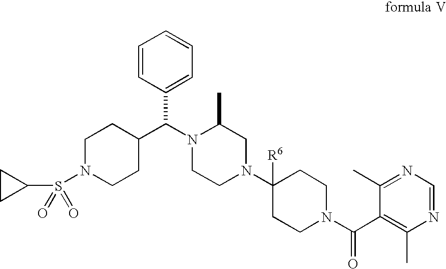
or a pharmaceutically acceptable salt of solvate thereof.

25. The pharmaceutical composition of claim 19, wherein the CXCR4 antagonist is at least one of AMD-070, CS-3955 KRH-1 120, KRH-2731, and KRH-1636.

26. The pharmaceutical composition of claim 25, wherein the CXCR4 antagonist is AMD-070.

27. The pharmaceutical composition of claim 25, wherein the CXCR4 antagonist is CS-3955.

28. The pharmaceutical composition of claim 25, wherein the CXCR4 antagonist is at least one of KRH-1120, KRH-2731, KRH-1636.

29. The pharmaceutical composition of claim 19, wherein the CCR5 antagonist is present in a therapeutically effective amount.

30. The pharmaceutical composition of claim 19, wherein the CXCR4 antagonist is present in a therapeutically effective amount.

31. A method of treating Human Immunodeficiency Virus comprising administering to a human in need of such treatment a therapeutically effective amount of a pharmaceutical composition of claim 19.

32. The method of claim 31, wherein the CCR5 antagonist is represented by formula I: 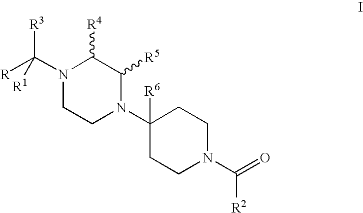
or a pharmaceutically acceptable salt or solvate thereof,
wherein R is optionally substituted phenyl, pyridyl, thiophenyl or naphthyl;
R¹ is hydrogen or alkyl;
R² is substituted phenyl, substituted heteroaryl, naphthyl, fluorenyl, diphenylmethyl or optionally substituted phenyl- or heteroaryl-alkyl;
R³ is hydrogen, alkyl, alkoxyalkyl, cycloalkyl, cycloalkylalkyl, or optionally substituted phenyl, phenylalkyl, naphthyl, naphthylalkyl, heteroaryl or heteroarylalkyl;
R⁴, R⁵ and R⁷ are hydrogen or alkyl; and
R⁶ is hydrogen, alkyl or alkenyl.

33. The method of claim 31, wherein the CCR5 antagonist is a compound of formula II: 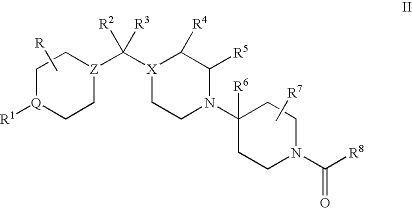
or a pharmaceutically acceptable salt, solvate, or ester thereof, wherein:
Q, X and Z are independently selected from the group consisting of CH and N, provided that one or both of Q and Z is N;
R, R⁴, R⁵, R⁶ and R⁷ are independently selected from the group consisting of H and (C₁-C₆)alkyl;
R¹ is H, (C₁-C₆)alkyl, fluoro-(C₁-C₆)alkyl-, R⁹-aryl(C₁-C₆)alkyl-, R⁹-heteroaryl-(C₁-C₆)alkyl-, (C₁-C₆)alkyl-SO₂—, (C₃-C₆)cycloalkyl-SO₂—, fluoro-(C₁-C₆)alkyl-SO₂—, R⁹-aryl-SO₂—, R⁹-heteroaryl-SO₂—, N(R²²)(R²³)-SO₂—, (C₁-C₆)alkyl-C(O)—, (C₃-C₆)cyclo-alkyl-C(O)—, fluoro-(C₁-C₆)alkyl-C(O)—, R⁹-aryl-C(O)—, NH—(C₁-C₆)alkyl-C(O)— or R⁹-aryl-NH—C(O)—;
R² is H or (C₁-C₆)alkyl, and R³ is H, (C₁-C₆)alkyl, (C₁-C₆)alkoxy(C₁-C₆)alkyl-, (C₃-C₁₀)-cycloalkyl-, (C₃-C₁₀)cycloalkyl(C₁-C₆)alkyl-, R⁹-aryl, R⁹-aryl(C₁-C₆)-alkyl-, R⁹-heteroaryl, or R⁹-heteroaryl(C₁-C₆)alkyl-, provided that both X and Z are not each N;
or R² and R³ together are ═O, ═NOR¹⁰, ═N—NR¹¹R¹² or ═CH(C₁-C₆)alkyl, provided that when one or both of X and Z is N, R² and R³ together are not ═CH(C₁-C₆)alkyl;
and when X and Z are each CH, R³ can also be (C₁-C₆)alkoxy, R⁹-aryloxy, R⁹-heteroaryloxy, (C₁-C₆)alkyl-C(O)O—, (C₁-C₆)alkyl-NH—C(O)O—, N((C₁-C₆)alkyl)₂—C(O)O—, (C₁-C₆)alkyl-C(O)—NR¹³13 , (C₁-C₆)alkyl-O—C(O)—NR¹³—, (C₁-C₆)alkyl-NH—C(O)—NR¹³— or N((C₁-C₆)alkyl)₂—C(O)— NR¹³—;
R⁸ is (R¹⁴,R¹⁵,R¹⁶)-substituted phenyl, (R¹⁴, R¹⁵, R¹⁶)-substituted 6-membered heteroaryl, (R¹⁴,R¹⁵,R¹⁶)-substituted 6-membered heteroaryl N-oxide, (R¹⁷,R¹⁸)-substituted 5-membered heteroaryl, naphthyl, fluorenyl, 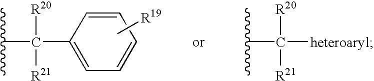
diphenylmethyl,
R⁹ is 1, 2 or 3 substituents independently selected from the group consisting of H, halogen, (C₁-C₆)alkyl, (C₁-C₆)alkoxy, —CF₃, —OCF₃, CH₃C(O)—, —CN, CH₃SO₂—, CF₃SO₂— and —N(R²²)(R²³);
R¹⁰ is H, (C₁-C₆)alkyl, fluoro(C₁-C₆)alkyl-, (C₃-C₁₀)cycloalkyl(C₁-C₆)alkyl-, hydroxy(C₂-C₆)alkyl-, (C₁-C₆)alkyl-O—(C₂-C₆)alkyl-, (C₁-C₆)alkyl-O—C(O)—(C₁-C₆)alkyl- or N(R²²)(R²³)—C(O)—(C₁-C₆)alkyl-;
R¹¹ and R¹² are independently selected from the group consisting of H, (C₁-C₆)alkyl and (C₃-C₁₀)cycloalkyl, or R¹¹ and R¹² together are C₂-C₆ alkylene and form a ring with the nitrogen to which they are attached;
R¹⁴ and R¹⁵ are independently selected from the group consisting of (C₁-C₆)alkyl, halogen, —NR²²R²³, —OH, —CF₃, —OCH₃, —O-acyl and —OCF₃;
R¹⁶ is R¹⁴, hydrogen, phenyl, —NO₂, —CN, —CH₂F, —CHF₂, —CHO, —CH═NOR²⁴, pyridyl, pyridyl N-oxide, pyrimidinyl, pyrazinyl, —N(R²⁴)CONR²⁵R²⁶, —NHCONH(chloro-(C₁-C₆)alkyl), —NHCONH((C₃-C₁₀)cycloalkyl(C₁-C₆)alkyl), —NHCO(C₁-C₆)alkyl, —NHCOCF₃, —NHSO₂N(R²²)(R²³), —NHSO₂(C₁-C₆)alkyl, —N(SO₂CF₃)₂, —NHCO₂-(C₁-C₆)alkyl, C₃-C₁₀ cycloalkyl, —SR²⁷, —SOR²⁷, —SO₂R²⁷, —SO₂NH(R²²), —OSO₂(C₁-C₆)alkyl, —OSO₂CF₃, hydroxy(C₁-C₆)alkyl-, —CON R²⁴R²⁵, —CON(CH₂CH₂OCH₃)₂, —OCONH(C₁-C₆)alkyl, —CO₂R²⁴, —Si(CH₃)₃ or —B(OC(CH₃)₂)₂;
R¹⁷ is (C₁-C₆)alkyl, —N(R²²)(R²³) or R¹⁹-phenyl;
R¹³, R¹⁸, R²², R²³, R²⁴, R²⁵ and R²⁶ are independently selected from the group consisting of H and (C₁-C₆)alkyl;
R¹⁹ is 1, 2 or 3 substituents independently selected from the group consisting of H, (C₁-C₆)alkyl, —CF₃, —CO₂R²⁵, —CN, (C₁-C₆)alkoxy and halogen;
R²⁰ and R²¹ are independently selected from the group consisting of H and (C₁-C₆)alkyl, or R²⁰ and R²¹ together with the carbon to which they are attached form a spiro ring of 3 to 6 carbon atoms; and
R²⁷ is (C₁-C₆)alkyl or phenyl.

34. The method of claim 32, wherein the CCR5 antagonist of formula I is a compound of formula III: 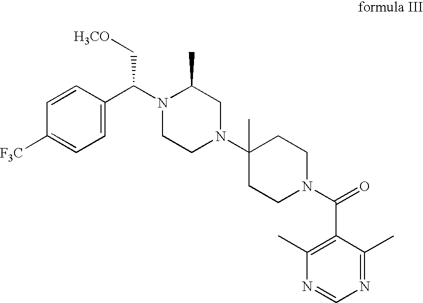
or a pharmaceutically acceptable salt or solvate thereof.

35. The method of claim 33, wherein the CCR5 antagonist of formula II is a compound of formula IV: 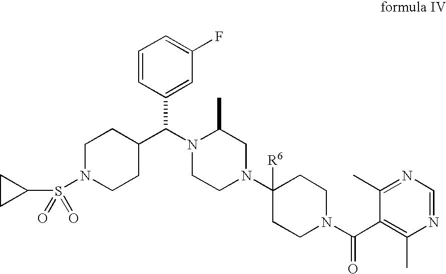
or a pharmaceutically acceptable salt or solvate thereof.

36. The method of claim 33, wherein the CCR5 antagonist of formula II is a compound of formula V: 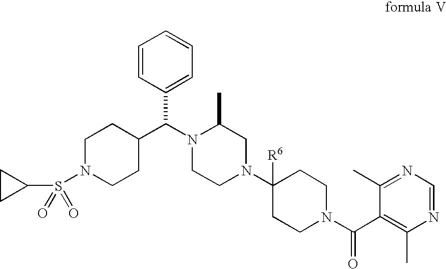
or a pharmaceutically acceptable salt of solvate thereof.

37. The method of claim 31, wherein the CXCR4 antagonist is at least one of AMD-070, CS-3955, KRH-1120, KRH-2731, and KRH-1636.

38. The method of claim 37, wherein the CXCR4 antagonist is AMD-070.

39. The method of claim 37, wherein the CXCR4 antagonist is CS-3955.

40. The method of claim 37, wherein the CXCR4 antagonist is at least one of KRH-1120, KRH-2731, and KRH-1636.

41. The method of claim 31, wherein the pharmaceutical composition is administered orally.

42. The method of claim 31, wherein the pharmaceutical composition is administered subcutaneously.

43. The method of claim 31, further comprising administering one or more antiviral or therapeutic agents useful in the treatment of HIV.

44. The method of claim 43, wherein the antiviral agent is at least one of reverse transcriptase inhibitors, non-nucleoside reverse transcriptase inhibitors, and protease inhibitors.

45. The method of claim 43, wherein the pharmaceutical composition and the one or more antiviral or therapeutic agents are sequentially administered.

46. The method of claim 43, wherein the pharmaceutical composition is administered before the one or more antiviral or therapeutic agents.

47. The method of claim 43, wherein the one or more antiviral or therapeutic agents is administered before the pharmaceutical composition.

48. The method of claim 43, wherein the pharmaceutical composition and the one or more antiviral or therapeutic agents are administered at the same time.

49. The method of claim 31, wherein the human is a treatment-naive patient.

50. The method of claim 31, wherein the human is a treatment-experienced patient.

51. A method of treating solid organ transplant rejection, graft v. host disease, arthritis, atopic dermatitis, psoriasis, asthma, allergies, inflammatory bowel disease, rheumatoid arthritis or multiple sclerosis comprising administering to a human in need of such treatment a therapeutically effective amount of a composition of claim 1.

52. A method of treating solid organ transplant rejection, graft v. host disease, arthritis, atopic dermatitis, psoriasis, asthma, allergies, inflammatory bowel disease, rheumatoid arthritis or multiple sclerosis comprising administering to a human in need of such treatment a therapeutically effective amount of a composition of claim 2.

53. A kit comprising, in separate containers:
a first container comprising a pharmaceutical composition comprising a therapeutically effective amount of a CCR5 antagonist, and a pharmaceutically acceptable carrier; and
a second container comprising a pharmaceutical composition comprising an effective amount of a CXCR4 antagonist and a pharmaceutically acceptable carrier.

54. The kit of claim 53, wherein the CCR5 antagonist is represented by formula I: 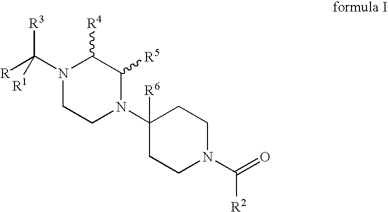
or a pharmaceutically acceptable salt or solvate thereof,
wherein R is optionally substituted phenyl, pyridyl, thiophenyl or naphthyl;
R¹ is hydrogen or alkyl;
R² is substituted phenyl, substituted heteroaryl, naphthyl, fluorenyl, diphenylmethyl or optionally substituted phenyl- or heteroaryl-alkyl;
R³ is hydrogen, alkyl, alkoxyalkyl, cycloalkyl, cycloalkylalkyl, or optionally substituted phenyl, phenylalkyl, naphthyl, naphthylalkyl, heteroaryl or heteroarylalkyl;
R⁴, R⁵ and R⁷ are hydrogen or alkyl; and
R⁶ is hydrogen, alkyl or alkenyl.

55. The kit of claim 53, wherein the CCR5 antagonist is represented by formula II: 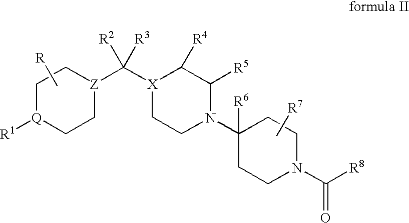
or a pharmaceutically acceptable salt, solvate, or ester thereof, wherein:
Q, X and Z are independently selected from the group consisting of CH and N, provided that one or both of Q and Z is N;
R, R⁴, R⁵, R⁶ and R⁷ are independently selected from the group consisting of H and (C₁-C₆)alkyl;
R¹ is H, (C₁-C₆)alkyl, fluoro-(C₁-C₆)alkyl-, R⁹-aryl(C₁-C₆)alkyl-, R⁹-heteroaryl-(C₁-C₆)alkyl-, (C₁-C₆)alkyl-SO₂—, (C₃-C₆)cycloalkyl-SO₂—, fluoro-(C₁-C₆)alkyl-SO₂—, R⁹-aryl-SO₂—, R⁹-heteroaryl-SO₂—, N(R²²)(R²³)-SO₂—, (C₁-C₆)alkyl-C(O)—, (C₃-C₆)cyclo-alkyl-C(O)—, fluoro-(C₁-C₆)alkyl-C(O)—, R⁹-aryl-C(O)—, NH—(C₁-C₆)alkyl^{C(O)— or R}⁹-aryl-NH—C(O)—;
R² is H or (C₁-C₆)alkyl, and R³ is H, (C₁-C₆)alkyl, (C₁-C₆)alkoxy(C₁-C₆)alkyl-, (C₃-C₁₀)-cycloalkyl-, (C₃-C₁₀)cycloalkyl(C₁-C₆)alkyl-, R⁹-aryl, R⁹-aryl(C₁-C₆)-alkyl-, R⁹-heteroaryl, or R⁹-heteroaryl(C₁-C₆)alkyl-, provided that both X and Z are not each N;
or R² and R³ together are ═O, ═NOR¹⁰, ═N—NR¹¹R¹² or ═CH(C₁-C₆)alkyl, provided that when one or both of X and Z is N, R² and R³ together are not ═CH(C₁-C₆)alkyl;
and when X and Z are each CH, R³ can also be (C₁-C₆)alkoxy, R⁹-aryloxy, R⁹-heteroaryloxy, (C₁-C₆)alkyl-C(O)O—, (C₁-C₆)alkyl-NH—C(O)O—, N((C₁-C₆)alkyl)₂—C(O)O—, (C₁-C₆)alkyl-C(O)—NR¹³—, (C₁-C₆)alkyl-O—C(O)—NR¹³—, (C₁-C₆)alkyl-NH—C(O)—NR¹³— or N((C₁-C₆)alkyl)₂—C(O)—NR¹³—;
R⁸ is (R¹⁴, R¹⁵,R¹⁶)-substituted phenyl, (R¹⁴, R¹⁵,R¹⁶)-substituted 6-membered heteroaryl, (R¹⁴,R¹⁵,R¹⁶)-substituted 6-membered heteroaryl N-oxide, (R ¹⁷,R¹⁸)-substituted 5-membered heteroaryl, naphthyl, fluorenyl, diphenylmethyl, 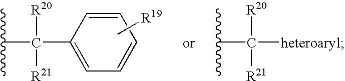
R⁹ is 1, 2 or 3 substituents independently selected from the group consisting of H, halogen, (C₁-C₆)alkyl, (C₁-C₆)alkoxy, —CF₃, —OCF₃, CH₃C(O)—, —CN, CH₃SO₂—, CF₃SO₂— and —N(R²²)(R²³);
R¹⁰ is H, (C₁-C₆)alkyl, fluoro(C₁-C₆)alkyl-, (C₃-C₁₀)cycloalkyl(C₁-C₆)alkyl-, hydroxy(C₂-C₆)alkyl-, (C₁-C₆)alkyl-O—(C₂-C₆)alkyl-, (C, —C₆)alkyl-O—C(O)—(C₁-C₆)alkyl- or N(R²²)(R²³)—C(O)—(C₁-C₆)alkyl-;
R¹¹ and R¹² are independently selected from the group consisting of H, (C₁-C₆)alkyl and (C₃-C₁₀)cycloalkyl, or R¹¹ and R¹² together are C₂-C₆ alkylene and form a ring with the nitrogen to which they are attached;
R¹⁴ and R¹⁵ are independently selected from the group consisting of (C₁-C₆)alkyl, halogen, —NR²²R²³, —OH, —CF₃, —OCH₃, —O-acyl and —OCF₃;
R¹⁶ is R¹⁴, hydrogen, phenyl, —NO₂, —CN, —CH₂F, —CHF₂, —CHO, —CH═NOR²⁴, pyridyl, pyridyl N-oxide, pyrimidinyl, pyrazinyl, —N(R²⁴)CONR²⁵R²⁶, —NHCONH(chloro-(C₁-C₆)alkyl), —NHCONH((C₃-C₁₀)cycloalkyl(C₁-C₆)alkyl), —NHCO(C₁-C₆)alkyl, —NHCOCF₃, —NHSO₂N(R²²)(R²³), —NHSO₂(C₁-C₆)alkyl, —N(SO₂CF₃)₂, —NHCO₂—(C₁-C₆)alkyl, C₃-C₁₀ cycloalkyl, —SR²⁷, —SOR²⁷, —SO₂R²⁷, —SO₂NH(R²²), —OSO₂(C₁-C₆)alkyl, —OSO₂CF₃, hydroxy(C₁-C₆)alkyl-, —CON R²⁴R²⁵, —CON(CH₂CH₂OCH₃)₂, —OCONH(C₁-C₆)alkyl, —CO₂R²⁴, —Si(CH₃)₃ or —B(OC(CH₃)₂)₂;
R¹⁷ is (C₁-C₆)alkyl, —N(R²²)(R²³) or R¹⁹-phenyl;
R¹³, R¹⁸, R²², R²³, R²⁴, R²⁵ and R²⁶ are independently selected from the group consisting of H and (C₁-C₆)alkyl;
R¹⁹ is 1, 2 or 3 substituents independently selected from the group consisting of H, (C₁-C₆)alkyl, —CF₃, —CO₂R²⁵, —CN, (C₁-C₆)alkoxy and halogen;
R²⁰ and R²¹ are independently selected from the group consisting of H and (C₁-C₆)alkyl, or R²⁰ and R²¹ together with the carbon to which they are attached form a spiro ring of 3 to 6 carbon atoms; and
R²⁷ is (C₁-C₆)alkyl or phenyl.

56. The kit of claim 55, wherein the CCR5 antagonist of formula I is a compound of formula III: 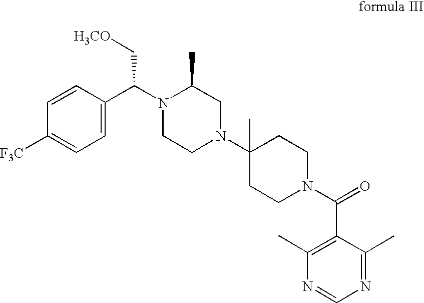
or a pharmaceutically acceptable salt or solvate thereof.

57. The kit of claim 55, wherein the CCR5 antagonist of formula II is a compound of formula IV: 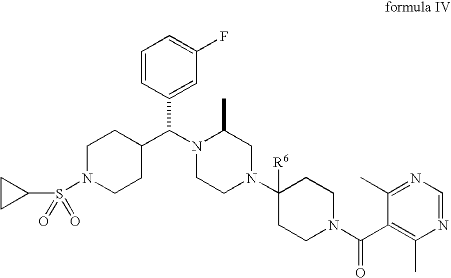
or a pharmaceutically acceptable salt or solvate thereof.

58. The kit of claim 55, wherein the CCR5 antagonist of formula II is a compound of formula V: 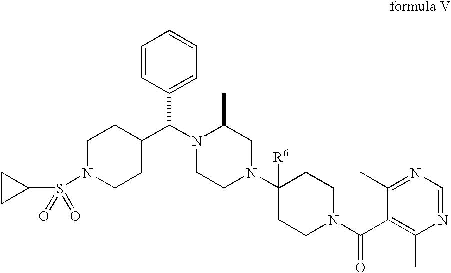
or a pharmaceutically acceptable salt of solvate thereof.

59. The kit of claim 53, wherein the CXCR4 antagonist is at least one of AMD-070, CS-3955, KRH-1120, KRH-2731, and KRH-1636.

60. The kit of claim 59, wherein the CXCR4 antagonist is AMD-070.

61. The kit of claim 59, wherein the CXCR4 antagonist is CS-3955.

62. The kit of claim 59, wherein the CXCR4 antagonist is at least one of KRH-1120, KRH-2731, KRH-1636.
